(19) United States
(12) Patent Application Publication
(10) Pub. No.: US 2020/0303068 A1
(43) Pub. Date: Sep. 24, 2020

(54) AUTOMATED TREATMENT GENERATION WITH OBJECTIVE BASED LEARNING

(71) Applicant: INTERNATIONAL BUSINESS MACHINES CORPORATION, Armonk, NY (US)

(72) Inventor: Takayuki Osogami, Kanagawa (JP)

(21) Appl. No.: 16/356,033

(22) Filed: Mar. 18, 2019

(51) Int. Cl. G16H 50/20; G16H 50/70; G06N 3/08; G06N 20/00

(52) U.S. Cl. CPC G16H 50/20; G06N 20/00; G06N 3/084; G16H 50/70

(57) ABSTRACT

Systems and methods for determining a treatment action include recording batches of data in a replay buffer, each of the batches including a present state, a previous state and a previous action. A value of each action in a set of candidate actions is evaluated at the present state according to a probability that each action achieves a goal of resolving a patient condition or achieves an objective for treating the patient condition by using a value model head corresponding to the goal and the objective. The treatment action is determined from the set of candidate actions according to the value of each action. The treatment action is communicated to a user to treat the patient condition. An error of the value of each action is determined according to whether the previous state achieved by the previous action matches the goal of the objective. Parameters of the value model are updated according to the error.

### BACKGROUND

The present invention generally relates to automated action generation with objective based learning, and more particularly to the prediction and generation of a healthcare actions for achieving a goal, such as, mitigating a condition, using objective-based learning.

Using reinforcement learning to achieve goals relies on rewarding a system (also known as an agent) according to whether a current state of the environment satisfies the goals. However, effectiveness can diminish where greater complexity is introduced because the agent can take a large quantity of actions prior to achieving the goal. For example, a patient treatment could be predicted according to reinforcement learning, and the treatment prediction agent can get rewarded for a long treatment plan even where some or many of the actions in the treatment plan are ineffective.

### SUMMARY

In accordance with an embodiment of the present invention, a method for determining a treatment action is presented. The method includes recording batches of data in a replay buffer, each of the batches including a present state, a previous state and a previous action. A value of each action in a set of candidate actions is evaluated at the present state according to a probability that each action achieves a goal of resolving a patient condition or achieves an objective for treating the patient condition by using a value model head corresponding to the goal and the objective. The treatment action is determined from the set of candidate actions according to the value of each action. The treatment action is communicated to a user to treat the patient condition. An error of the value of each action is determined according to whether the previous state achieved by the previous action matches the goal of the objective. Parameters of the value model are updated according to the error.

In accordance with another embodiment of the present invention, a method for determining a treatment action is presented. The method includes recording batches of data, each of the batches including a present state, a previous state and a previous action. A value of each action in a set of candidate actions is evaluated at the present state according to a probability that each action achieves a goal of resolving a patient condition or achieves one of a plurality of objectives for treating the patient condition by using a plurality of value model heads corresponding to each of the plurality of objectives and with a goal value model head corresponding to the goal. The treatment action is determined from the set of candidate actions according to the value of each action. The treatment action is communicated to a user to treat the patient condition. Parameters of a state representation model for achieving the objective are updated according to the value using a terminal difference error to perform reinforcement learning.

In accordance with another embodiment of the present invention, a system for determining a treatment action is presented. The system includes a replay buffer to record batches of data, each of the batches including a present state, a previous state and a previous action. A value model head corresponding to an objective for treating a patient condition evaluates a value of each action in a set of candidate actions at the present state according to a probability that each action achieves a goal of resolving a patient condition or achieves an objective for treating the patient condition. An optimizer determines the treatment action of the set of candidate actions according to the value of each action and to update parameters of a state representation model for achieving the objective according to the value determined by the value model. A connection communicates the treatment action to a user to treat the patient condition.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a diagram showing a treatment system for objective-based patient treatment, in accordance with an embodiment of the present invention;

FIG. 2 is a diagram showing a treatment agent that interacts with a condition monitor to learn patient treatment procedures according to objectives for achieving a goal, in accordance with an embodiment of the present invention;

FIG. 3 is a diagram showing a treatment agent that utilizes states and actions with a state representation model and value model to predict treatment procedure and assess rewards for objectives, in accordance with an embodiment of the present invention;

FIG. 4 is a diagram showing a state representation model with multiple layers with optimization from a multi-head value model, in accordance with an embodiment of the present invention;

FIG. 5 is a generalized diagram showing a neural network, in accordance with an embodiment of the present invention;

FIG. 6 is a diagram showing an artificial neural network (ANN) architecture, in accordance with an embodiment of the present invention;

FIG. 7 is a block diagram showing neuron, in accordance with an embodiment of the present invention;

FIG. 8 is a block diagram showing an exemplary processing system in accordance with one embodiment;

FIG. 9 is a block diagram showing an illustrative cloud computing environment having one or more cloud computing nodes with which local computing devices used by cloud consumers communicate in accordance with one embodiment;

FIG. 10 is a block diagram showing a set of functional abstraction layers provided by a cloud computing environment in accordance with one embodiment; and

FIG. 11 is a block/flow diagram showing a system/method generating objective-based treatment actions, in accordance with an embodiment of the present invention.

### DETAILED DESCRIPTION

According to an embodiment of the present invention, a reinforcement learning agent is presented that utilizes rewards based on intermediate objectives, even where an ultimate goal has not yet been achieved. Because many tasks result in a variety of actions to achieve a goal, the agent can be more accurate and efficiently trained where actions are rewarded for achieving objectives.

Intermediate objectives are a set of sub-goals that contribute to achieving an end-goal. For example, according to one possible embodiment, the agent is designed to predict patient treatments to satisfy the end-goal of achieving patient health via, e.g., curing a disease, improving biomarkers, mitigating symptoms, or other end goal of a treatment pathway. The end-goal of, e.g., curing the patient, can include intermediate objectives such as, e.g., reducing high blood pressure, improving hormone balances, achieving optimal white blood cell counts, achieving optimal weight and nutrition, among other health related objectives.

The intermediate objectives are implemented with independent heads of a learning network while the end-goal has a global head. As a result, the agent receives rewards from a head when a corresponding objective is attained. All heads provide a reward when the end-goal is attained. Thus, the agent is trained based on attaining intermediate objectives along the way to attaining the goal. While it would be possible to configure the intermediate objectives as separate goals, with a reward received for each goal, such an approach can obfuscate the actual end-goal to be achieved by providing rewards across divergent goals. Using a goal with intermediate objective approach, as described, facilitates learning to achieve the actual goal, with additional feedback from achieving the objectives. Thus, training efficiency and prediction accuracy are improved.

Exemplary applications/uses to which the present invention can be applied include, but are not limited to: reinforcement learning based predictions, such as, e.g., game theory prediction, control systems, disease treatment, financial management, sales automation, among others.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

An artificial neural network (ANN) is an information processing system that is inspired by biological nervous systems, such as the brain. The key element of ANNs is the structure of the information processing system, which includes a large number of highly interconnected processing elements (called “neurons”) working in parallel to solve specific problems. ANNs are furthermore trained in-use, with learning that involves adjustments to weights that exist between the neurons. An ANN is configured for a specific application, such as pattern recognition or data classification, through such a learning process.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

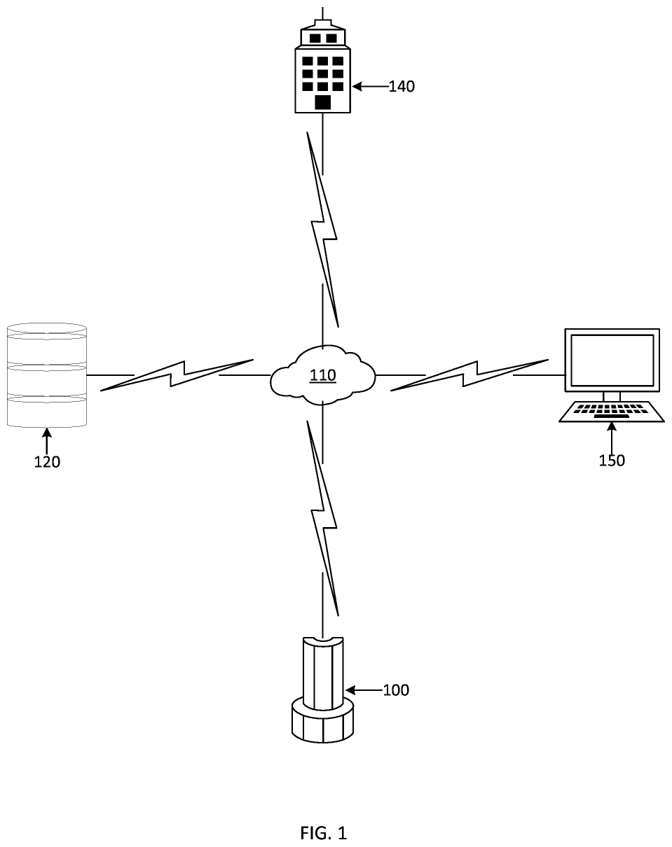

Referring now to the drawings in which like numerals represent the same or similar elements and initially to FIG. 1, a diagram showing a treatment system for objective-based patient treatment is depicted according to an embodiment of the present invention.

According to one possible embodiment of the present invention, a treatment prediction agent 100 can communicate with a network 110 to receive data and generate treatment procedure predictions, such as, e.g., medication dosing, prescribing therapy interventions and scheduling, scheduling care visits and consultations, providing diet and fitness advice, among other medical interventions.

The treatment prediction agent 100 receives the data from, e.g., a database 120 or a care center 140 such as a hospital. The data can include, e.g., any patient health data useable for determining a diagnosis and treatment such as, blood pressure, heart rate, age, height, weight, injuries, white blood cell count, red blood cell count, blood oxygen levels, calorie intake, fitness level, sleep patterns, among other biomarkers and health data and histories thereof. The data can be collected at a care center 140 and provided directly to the treatment prediction agent 100, or stored in the database 120 for later retrieval by the treatment prediction agents 100.

The treatment prediction agent 100 can retrieve the data and formulate treatment actions for a patient. Treatment actions can include, e.g., any suitable medical intervention for treating an adverse condition. For example, the care center 140 and/or the database 120, or other connected system, can determine an adverse condition in a patient according to the data. Alternatively, a health care professional can manually provide a pre-diagnosed adverse condition corresponding to the patient at a user access terminal 150. The user access terminal 150 can be, e.g., a thin client for connecting to the network 110, a personal computer, a mobile device such as a smartphone, tablet or personal digital assistant, or other device facilitating user interaction across the network 110 with the database 120, care center 140 and treatment prediction agent 100. The adverse condition can be provided across the network 110 to the treatment agent 100 to determine a resolution the adverse condition. As such, the resolution of adverse condition is a goal of the treatment prediction agent 100.

Upon predicting a treatment action, the treatment prediction agent 100 can provide the action to a healthcare professional, such as a doctor or nurse, at the care center 140 and/or via the user access terminal 150. Alternatively, the treatment prediction agent 100 can provide treatment actions directly to a patient via the user access terminal 150 in the form of, e.g., exercise advice, diet advice, among other healthcare advice to meet health goals of an individual.

The treatment prediction agent 100 predicts treatment actions for an episode of treatment, such as, e.g., a specified time-period. The treatment actions can be, e.g., rewarded through reinforcement learning techniques to optimize the treatment prediction agent 100 methodology. For example, the treatment prediction agent 100 can include, e.g., a neural network, such as, a convolutional neural network or recurrent neural network, for representing a current state of a patient. The treatment prediction agent 100 can then use the current state to evaluate each action of a set of actions to predict an appropriate action to treat the adverse conditions according to the evaluation. Reinforcement learning can be incorporated into the evaluation mechanism to update parameters of the treatment prediction agent 100 based on changes to the health of the patient. However, other prediction techniques may also be used.

The treatment prediction agent 100 can evaluate actions with reference to, not only the end goal of resolving the adverse condition, but also with reference to other objectives that can lead to the resolution of the adverse condition. For example, a greater value can be determined for an increased likelihood in an action of the set of actions resulting in, e.g., decreased blood pressure, improved resting heart rate, improved weight, decreased coughing, or other beneficial effect to biomarkers and health indicia. Moreover, a previous action can be used to provide positive reinforcement through reinforcement learning for the use an action that resulted in an objective being attained. Reinforcement, as well as an evaluation of actions in the set of actions can be performed concurrently.

In particular, the treatment agent 100 can suggest a treatment action according to an evaluation of the set of actions. The suggested treatment action as well as a measured state in response to the suggested treatment action can be provided back to the treatment agent 100. The degree of success of the suggested treatment action can be evaluated while also evaluating the set of actions to suggest a new treatment action in light of the measured state. The degree of success of the suggested treatment action is used to provide reinforcement to the treatment agent 100. The reinforcement can come from the degree of success of achieving objectives, including beneficial effects to biomarkers and health indicia, that are specifically related to the adverse condition of the patient. Moreover, the treatment predication agent 100 receives a reward for actions that ultimately do lead to the resolution of the adverse condition.

The actions of the treatment prediction agent 100 can be periodically assessed for success relative to the goal or to an objective. For example, the treatment prediction agent 100 can be assessed at the end of every episode, such as, e.g., every week, every month or other amount of time. Alternatively, the treatment prediction agent 100 can be assessed after each action, or upon resolution of the adverse condition in a patient. Once assessed, the treatment prediction agent 100 can receive rewards for achieving any of the goal or the objectives, and update parameters accordingly.

Because the treatment prediction agent 100 is updated based on both the goal and the objectives that lead to achieving the goal, the treatment prediction agent 100 can receive reinforcement on a more directed fashion that facilitates training even where the goal has yet to be achieved. Thus, actions taken in episodes prior to the ultimate resolution of the condition can be correlated with effectiveness, and the treatment prediction agent 100 can be trained accordingly. Thus, predictions can be made more efficient and accurate by providing more metrics for training metrics that direct training towards the goal even prior to achieving the goal.

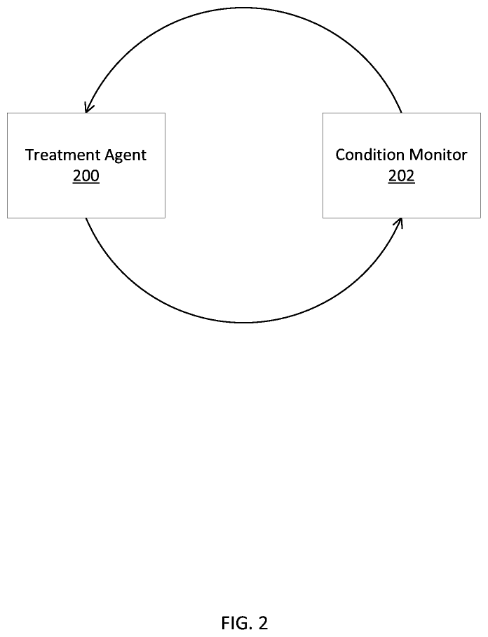

Referring now to FIG. 2, a diagram showing a treatment agent that interacts with a condition monitor to learn patient treatment procedures according to objectives for achieving a goal is depicted according to an embodiment of the present invention.

According to an embodiment of the present invention, a treatment agent 200 can interact with a condition monitor 202 for feedback on actions taken in treating a patient. The treatment agent 200 can suggest an action to take to treat an adverse condition of the patient. The condition monitor 202 can implement the action, or record biological effects upon the implementation of the action by a healthcare professional. Thus, the condition monitor 202 can include a medical instrument, such as, e.g., a blood pressure cuff, a heart rate sensor, a blood oxygen sensor, a scale, a blood test, or other medical measurements and combinations thereof.

The condition monitor 202 can assess the patient for changes to biomarkers and health indicia as a result of the action. The changes can be used to make a state determination of the adverse condition of the patient. The state determination can be performed by the condition monitor 202 and then provided to the treatment agent 200. However, according an embodiment of the present invention, the changes can be provided to the treatment agent 200 and the treatment agent 200 can perform the state determination by, e.g., encoding the changes with a state representation network to generate a feature vector corresponding to the measured changes.

Based upon the state determination and the action taken, the treatment agent 200 assess the effectiveness of the action based upon, e.g., a value function or other reinforcement learning or machine learning technique. Where the action is deemed effective, the treatment agent 200 can be rewarded. The treatment agent 200 can also be punished for ineffective actions. According to at least one embodiment, the effectiveness of an action can be determined by, e.g., comparison to the goal of curing the adverse condition and/or achieving objectives corresponding to the adverse condition.

The treatment agent 200 can then be adjusted to take into account the effectiveness or ineffectiveness of the action by, e.g., updating parameters corresponding to a state representation model and a value model. Additionally, the treatment agent 200 also determines a value for each possible action to take at a next step in response to the current measured state of the patient. According to the values for each action, a next action can be determined and suggested to a user. The treatment agent 200 can continue generating actions until a state corresponding to a resolution of the adverse condition is reached.

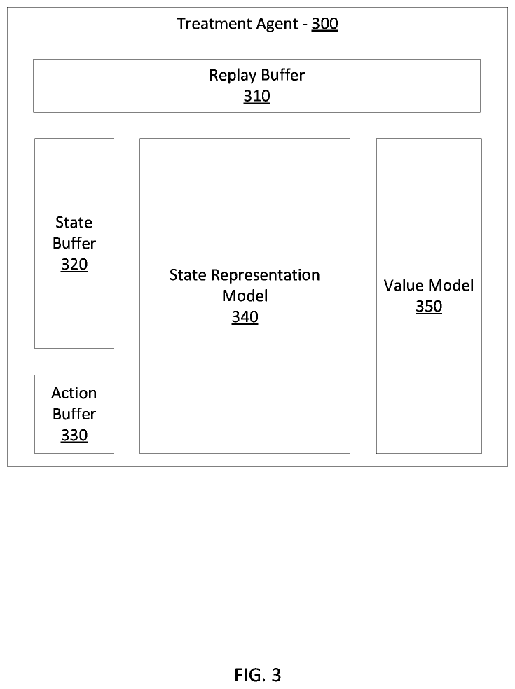

Referring now to FIG. 3, a diagram showing a treatment agent that utilizes states and actions with a state representation model and value model to predict treatment procedure and assess rewards for objectives is depicted according to an embodiment of the present invention.

According to an embodiment of the present invention, a treatment agent 300 determines a treatment pathway, including treatment actions based on the health state of a patient, including, e.g., a diagnosis of an adverse condition. To generate the pathway, the treatment agent 300 predicts an action at a current time frame and analyzes a change to a state of the patient as a result of that action. The treatment pathway is progressively formed through action generation, such as, discrete actions to treat the adverse condition, or a treatment protocol for a given period of time. The new state resulting from the actions can then be measured after, e.g., the discrete action or the period of time for the protocol. The treatment agent 300 can be trained according to whether the new state matches an objective or the goal of resolving the adverse condition.

The treatment agent 300 employs a replay buffer 310 to record and log batches of treatment data. For example, the replay buffer 310 can, e.g., record actions, states and goal and objective statuses, among other treatment data. The replay buffer 310 can receive an action selected on the basis of the outputs from the value model 350, and a new state from an environment such as, e.g., the condition monitor 202 described above. For example, a batch of data in the replay buffer can include, e.g.: (s, a, s′, r, r^{o}^{₁}, . . . , r^{o}^{ₙ}), where s is a previous state, a is a previous action, s′ is a new state, r is a goal status, and r^{o}^{₁} through r^{o}^{ₙ} are objective statuses. Thus, a batch of data in the replay buffer can include information relevant to both the state representation model 340 for a representation for the current state, as well as the value model 350 for evaluation of an action at the current state.

A batch from a prior time frame can be used for prediction. As a result, a current state, a prior state and a prior action can be fed from the replay buffer 310 as a batch to the state representation model 340 and value model 350. To facilitate feeding the batch, a state buffer 340 can provide a cache of states near the state representation model 340 and value model 350 for temporary efficient storage of data. Similarly, previous actions can be fed via an action buffer 330 to efficiently handle action data. The replay buffer 310, state buffer 320 and action buffer 330 can each include, e.g., volatile or non-volatile memory such as, e.g., random access memory (RAM), virtual RAM (vRAM), flash storage, cache, or other temporary storage solution for buffering data to the state representation model 340.

The state representation model 340 can access the data from the state buffer 320 and the action buffer 330 to determine a representation for the current state, which in turn will be used for evaluating actions for, e.g., treatment of the adverse condition. For example, the state representation model 340 can retrieve a current state from the state buffer 320 and generate a representation for the current state using, e.g., a neural network such as a convolutional neural network (CNN), a recurrent neural network (RNN), a deep neural network (DNN), or other suitable neural network for generate an action prediction in response to an observed state. The state representation model 340 can also incorporate a past state and a past action to provide more information to determine the representation for the current state, thus improving accuracy.

Each action can be assessed against a newly measured state using the value model 350. The newly measured state can be provided by, e.g., a condition monitoring device such as, e.g., the condition monitor 202 described above. The new state, the previous state, each action and a comparison of the new state with the goal and objectives can be used to determine a value of each action. The value corresponds to a quantitative measurement of the action's contribution towards achieving the goal and the objectives.

Any number of objectives may be used that correspond to the goal, such as, e.g., objectives that facilitate achieving resolution of the adverse condition. As such, the states resulting from predicted actions can be compared against, e.g., biomarker measurements corresponding to improvements in health related to the adverse condition. By using both the goal and objectives, the value model 350 can better train the parameters of the state representation model 340 by recognizing positive actions even where the adverse condition has not yet be resolved. As objectives are reached according to new states, the value model 350 provides rewards to the state representation model 340 to update the parameters of the state representation model 340 for subsequent actions according to, e.g., equation 1 below:

θ←θ+ηΣ_{(s,a,s′,r)}(TD(s,a,s′,r)∇Q_{θ}(s,a)), Equation 1

where θ is the state representation model parameters, η is a forgetting rate, TD refers to a temporal difference function, and Q is a value function. Thus, the value model 350 implements an equation such as, e.g., equation 1 above, to back propagate rewards to improve state representation model 340 parameters θ.

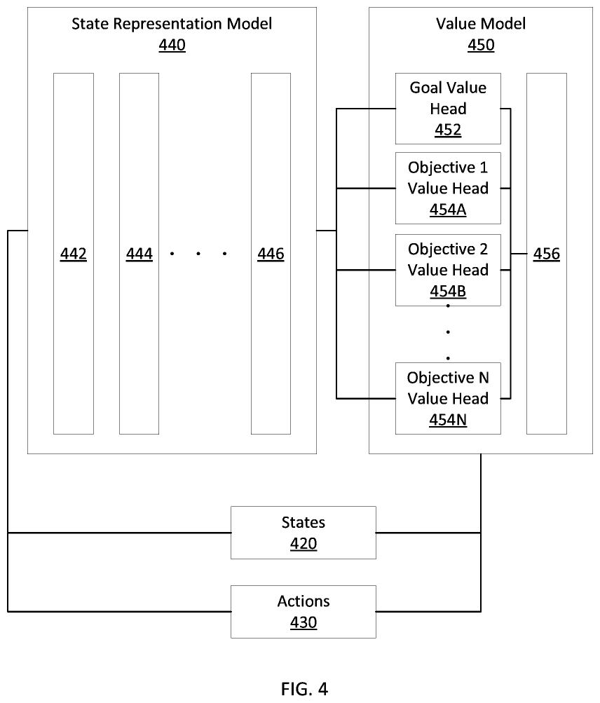

Referring now to FIG. 4, a diagram showing a state representation model with multiple layers with optimization from a multi-head value model is depicted according to an embodiment of the present invention.

According to an embodiment of the present invention, a state representation model 440 is in communication with a multi-head value model 450 to evaluate actions 430 and states 420 to optimize parameters of the state representation model 440. As such, a state 420 can be communicated to the state representation model 440. The state representation model 440 generates a feature vector of the current state 420 by, e.g., encoding the state 420 into the feature vector. Each new state 420 can be encoded by the prediction as it is received from, e.g., a condition monitor, such as, e.g., the condition monitor 202 described above. The feature vector generated by the state representation model 440 is then provided to the value model 450 to compare each of a set of candidate actions 430 with objectives given the encoded states 420, including the new state, and the goal and objectives corresponding to, e.g., treatment of an adverse condition of a patient.

The state representation model 440 can utilize a neural network such as, e.g., a DNN including a CNN. As such, the state representation model 440 can utilize parameters governing neural network layers 442, 444 through 446. While only three layers are depicted, the state representation model 440 can include any suitable number of layers for representation of the current state. The state representation model 440, including each layer 442, 444 through 446, can take into account a batch of data related to a current time frame. Such a batch can include, e.g., a current and previous state from a set states 420, a previous action from a set of actions 430, as well as goal and objective statuses. Upon processing by the final layer 446, the state representation model 440 outputs a representation for the current state including, e.g., a feature vector corresponding to an adverse condition.

The feature vector is evaluated by the value model 450, which incorporates reinforcement learning via a value function to update parameters. The value model 450 evaluates each action in the set of candidate actions 430 given the encoded state 420 and a previously encoded state according to a set of objectives for, e.g., treating the adverse condition, as well as the end goal of resolving the adverse condition. The value model 450 independently evaluates the value of each action with reference to each objective and the goal. However, rather than evaluating the actions with respect to the goal independently from the evaluation with respect to the objectives, the value model 450 evaluates the action with respect to the goal on a global basis. Thus, evaluation for each objective includes an evaluation with respect to the goal.

In other words, a value of each action with reference to a particular objective can be increased by either achieving the objective, or by achieving the goal. As a such, the value model 450 can include a value determination that corresponds to each objective, where the value determination is influenced by the state of the goal. Thus, a reward can be determined for an objective where the goal is met but the objective is not.

As a result, the value model 450 incorporates a multi-head configuration. Each head of the value model 450 can evaluate each action of the candidate actions 430 given the encoded state with respect to a corresponding objective and the goal. Thus, the value model 450 includes a goal value head 452 in addition to objective 1 value head 454A, objective 2 value head 454B through objective N value head 454N. The number of objective value heads matches the number of objectives within the set of objectives corresponding to the goal of, e.g., treating the adverse condition.

The goal value head 452 evaluates each action and the encoded state with respect to the end goal of, e.g., resolving the adverse condition of the patient. Thus, the action and state are compared with the goal of a resolved adverse condition. The value of each action corresponds to a probability that the action resolves the adverse condition. Where a given action is determined by the goal value head 452 as being likely to correspond to a successfully attained goal, a reward corresponding to the determined likelihood is generated to train model parameters. A reward is also generated according to whether the previous action met the goal according to a previously encoded state. The goal value head 452, therefore, can incorporate a predicted value of each action according to the present state as well as the success of the previous action to determine a value of each action according to the present model parameters. The model parameters can, therefore, be updated based on the success or lack thereof of the previous action using, e.g., a temporal difference error determination, such as, e.g., by equation 2 below:

$$T D ( s , a , {s}^{′} , r ) ≡ {Q}_{θ} ( s , a ) - ( r ( s , a ) + 1 ( {s}^{′} ) {Q}_{t a r} ( {s}^{′} , {a}^{′} ) ) , \qquad Equation 2$$

where Q_{θ} is a partial value function with the model parameters θ according to the previous state s and previous action a, r denotes the status of the goal where r is one if and only if the goal is satisfied and zero otherwise, I refers to the status of an episode where I is one if and only if the episode has not yet elapsed and is zero otherwise, and Qₜₐᵣ is the target value function of an action a′ of the set of candidate actions [symbol] with respect to the goal under target model parameters. Here, the episode refers to a predetermine time period for, e.g., treating the adverse condition and the partial value function Q determines the probability that the goal is attained based on the encoded state. Thus, equation 2 incorporates evaluation of the action and state with respect to the goal, and determine a temporal difference error accordingly.

Similarly, each objective value head 454A-454N can determine a temporal difference error according to the probability of each action of the candidate actions 430 at the present encoded state achieving a corresponding objective. However, the objective value heads 454A-454N also incorporate a reward for an action achieving the goal. Thus, where an action of the candidate actions 430 carries a given probability of either the goal or the objective of a corresponding objective value head 454A, 454B or 454N, a reward is increased for the temporal difference error corresponding to the given probability determined by a partial value function, such as, e.g., in equation 3 below:

$$T {D}^{o} ( s , a , {s}^{′} , {r}^{o} ) ≡ {Q}_{θ}^{o} ( s , a ) - ( {r}^{o} ( s , a ) + {I}^{o} ( {s}^{′} ) {Q}_{t a r}^{o} ( {s}^{′} , {a}^{′} ) + {I}_{o} ( {s}^{′} ) {Q}_{t a r} ( {s}^{′} , {a}^{′} ) ) , \qquad Equation 3$$

where TD^{o} is the temporal difference of an objective o, Q_{θ}^{o} is the partial value function of the state representation model 440 parameters θ corresponding to the objective o, r^{o} is the reward for the objective o, 1⁰ is the relevance of the objective o according to the objective o not being obtain but remaining relevant to the end goal, Qₜₐᵣ is the target partial value function for an action a′ of the set of candidate actions [symbol] with respect to the objective o, and 1ₒ is the relevance of the end goal according to the end goal being achieved or the objective o becoming otherwise irrelevant.

An optimization module 456 uses cumulative temporal difference error to update the parameters θ of the state representation model 440. Thus, the state representation model 440 can be updated and trained according to the changing states resulting from prediction actions. To train the parameters θ, the optimization module 456 can employ an optimization based on equation 1 above, such as, e.g., with equation 4 below:

θ←θ+ηΣ(_{s,a,s}_{¹}_{, r,r}_{⁰} _{1, . . . , r}_{^{O}}_{n)ϵβ}(TD(s,a,s¹, r)∇Q_{θ}(s,a)+Σ₀TD⁰(s,a,s¹, r⁰)∇Q_{θ}⁰(s,a). Equation 4

Accordingly, each action can be assessed according to a change in state of, e.g., the patient. The value model 450 can be trained to recognize higher value actions at each state of the patient by updating the model parameters θ, and determining the value of each action of the set of candidate actions according to each value head for each objective and the goal. The training of the value model 450 improves the accuracy and efficiency through reinforcement learning that takes into account sub-objectives corresponding to achieving a goal. The use of the objectives improves feedback to the state representation model 440, thus making training more efficient and accurate. As a result, while an embodiment of the present invention envisions applications to healthcare and treating patient conditions, the state representation model 440 and value model 450 can also be adapted other applications, including, e.g., automated video game opponents, automated sales and marketing systems, or other goal oriented tasks to improve reinforcement learning for achieving the goals.

Additionally, the value model 450 can receive each action and each encoded state to determine the value of each action with respect to the goal and to the objectives under the current model parameters θ. The value model 450 can select the greatest value action at the present encoded state to determine the action that is most likely to beneficially progress treatment such that resolution of the adverse condition is made more likely. For example, the optimization module 456 can determine the maximum value action in the set of actions according to the value of each action with respect to each objective and the goal. For example, the optimization module 456 can utilize maximization, including, e.g., equation 5, below:

$$\hat{a} = arg {max}_{a} {Q}_{θ} ( s , a ) * {\prod }_{i = 1}^{n} {Q}_{θ}^{i} ( s , a ) , \qquad Equation 5$$

where â is the highest value action with respect to achieving the goal and i is an index referring to the objective. The highest value action can then be provided to a user, such as, e.g., a healthcare professional as a recommended treatment action for the adverse condition of a particular patient. The highest value action can be provided to the user with a display, such as the user access terminal 150 described above.

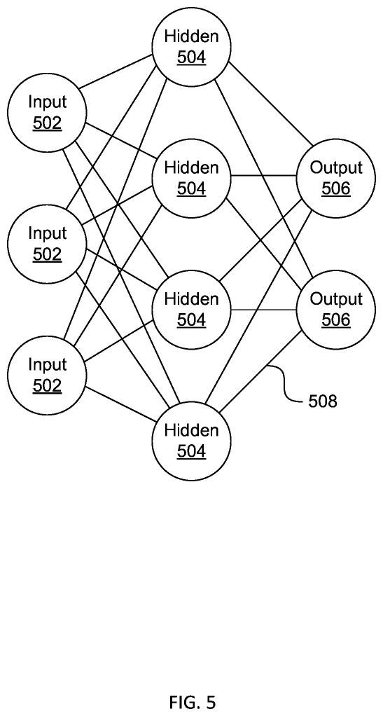

Referring now to FIG. 5, a generalized diagram of a neural network is shown. ANNs demonstrate an ability to derive meaning from complicated or imprecise data and can be used to extract patterns and detect trends that are too complex to be detected by humans or other computer-based systems. The structure of a neural network is known generally to have input neurons 502 that provide information to one or more “hidden” neurons 504. Connections 508 between the input neurons 502 and hidden neurons 504 are weighted and these weighted inputs are then processed by the hidden neurons 504 according to some function in the hidden neurons 504, with weighted connections 508 between the layers. There can be any number of layers of hidden neurons 504, and as well as neurons that perform different functions. There exist different neural network structures as well, such as convolutional neural network, maxout network, etc. Finally, a set of output neurons 506 accepts and processes weighted input from the last set of hidden neurons 504.

This represents a “feed-forward” computation, where information propagates from input neurons 502 to the output neurons 506. Upon completion of a feed-forward computation, the output is compared to a desired output available from training data. The error relative to the training data is then processed in “feed-back” computation, where the hidden neurons 504 and input neurons 502 receive information regarding the error propagating backward from the output neurons 506. Once the backward error propagation has been completed, weight updates are performed, with the weighted connections 508 being updated to account for the received error. This represents just one variety of ANN.

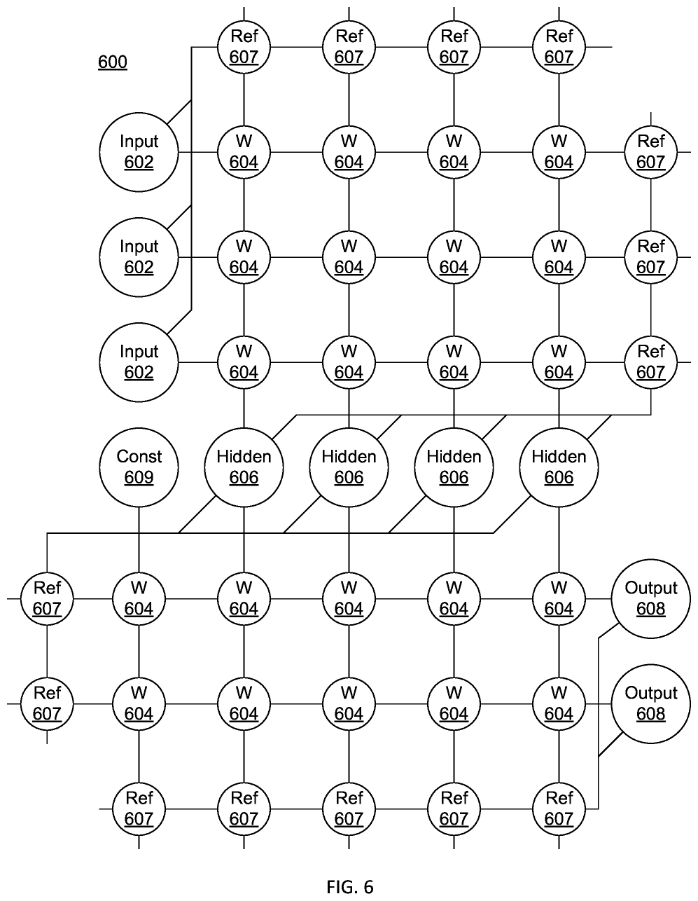

Referring now to the drawings in which like numerals represent the same or similar elements and initially to FIG. 6, an artificial neural network (ANN) architecture 600 is shown. It should be understood that the present architecture is purely exemplary and that other architectures or types of neural network can be used instead. In particular, while a hardware embodiment of an ANN is described herein, it should be understood that neural network architectures can be implemented or simulated in software. The hardware embodiment described herein is included with the intent of illustrating general principles of neural network computation at a high level of generality and should not be construed as limiting in any way.

Furthermore, the layers of neurons described below and the weights connecting them are described in a general manner and can be replaced by any type of neural network layers with any appropriate degree or type of interconnectivity. For example, layers can include convolutional layers, pooling layers, fully connected layers, stopmax layers, or any other appropriate type of neural network layer. Furthermore, layers can be added or removed as needed and the weights can be omitted for more complicated forms of interconnection.

During feed-forward operation, a set of input neurons 602 each provide an input voltage in parallel to a respective row of weights 604. In the hardware embodiment described herein, the weights 604 each have a settable resistance value, such that a current output flows from the weight 604 to a respective hidden neuron 606 to represent the weighted input. In software embodiments, the weights 604 can simply be represented as coefficient values that are multiplied against the relevant neuron outputs.

Following the hardware embodiment, the current output by a given weight 604 is determined as

$$I = \frac{V}{r} , \qquad$$

where V is the input voltage from the input neuron 602 and r is the set resistance of the weight 604. The current from each weight adds column-wise and flows to a hidden neuron 606. A set of reference weights 607 have a fixed resistance and combine their outputs into a reference current that is provided to each of the hidden neurons 606. Because conductance values can only be positive numbers, some reference conductance is needed to encode both positive and negative values in the matrix. The currents produced by the weights 604 are continuously valued and positive, and therefore the reference weights 607 are used to provide a reference current, above which currents are considered to have positive values and below which currents are considered to have negative values. The use of reference weights 607 is not needed in software embodiments, where the values of outputs and weights can be precisely and directly obtained. As an alternative to using the reference weights 607, another embodiment can use separate arrays of weights 604 to capture negative values.

The hidden neurons 606 use the currents from the array of weights 604 and the reference weights 607 to perform some calculation. The hidden neurons 606 then output a voltage of their own to another array of weights 604. This array performs in the same way, with a column of weights 604 receiving a voltage from their respective hidden neuron 606 to produce a weighted current output that adds row-wise and is provided to the output neuron 608.

It should be understood that any number of these stages can be implemented, by interposing additional layers of arrays and hidden neurons 606. It should also be noted that some neurons can be constant neurons 609, which provide a constant output to the array. The constant neurons 609 can be present among the input neurons 602 and/or hidden neurons 606 and are only used during feed-forward operation.

During back propagation, the output neurons 608 provide a voltage back across the array of weights 604. The output layer compares the generated network response to training data and computes an error. The error is applied to the array as a voltage pulse, where the height and/or duration of the pulse is modulated proportional to the error value. In this example, a row of weights 604 receives a voltage from a respective output neuron 608 in parallel and converts that voltage into a current which adds column-wise to provide an input to hidden neurons 606. The hidden neurons 606 combine the weighted feedback signal with a derivative of its feed-forward calculation and stores an error value before outputting a feedback signal voltage to its respective column of weights 604. This back propagation travels through the entire network 600 until all hidden neurons 606 and the input neurons 602 have stored an error value.

During weight updates, the input neurons 602 and hidden neurons 606 apply a first weight update voltage forward and the output neurons 608 and hidden neurons 606 apply a second weight update voltage backward through the network 600. The combinations of these voltages create a state change within each weight 604, causing the weight 604 to take on a new resistance value. In this manner the weights 604 can be trained to adapt the neural network 600 to errors in its processing. It should be noted that the three modes of operation, feed forward, back propagation, and weight update, do not overlap with one another.

As noted above, the weights 604 can be implemented in software or in hardware, for example using relatively complicated weighting circuitry or using resistive cross point devices. Such resistive devices can have switching characteristics that have a non-linearity that can be used for processing data. The weights 604 can belong to a class of device called a resistive processing unit (RPU), because their non-linear characteristics are used to perform calculations in the neural network 600. The RPU devices can be implemented with resistive random access memory (RRAM), phase change memory (PCM), programmable metallization cell (PMC) memory, or any other device that has non-linear resistive switching characteristics. Such RPU devices can also be considered as memristive systems.

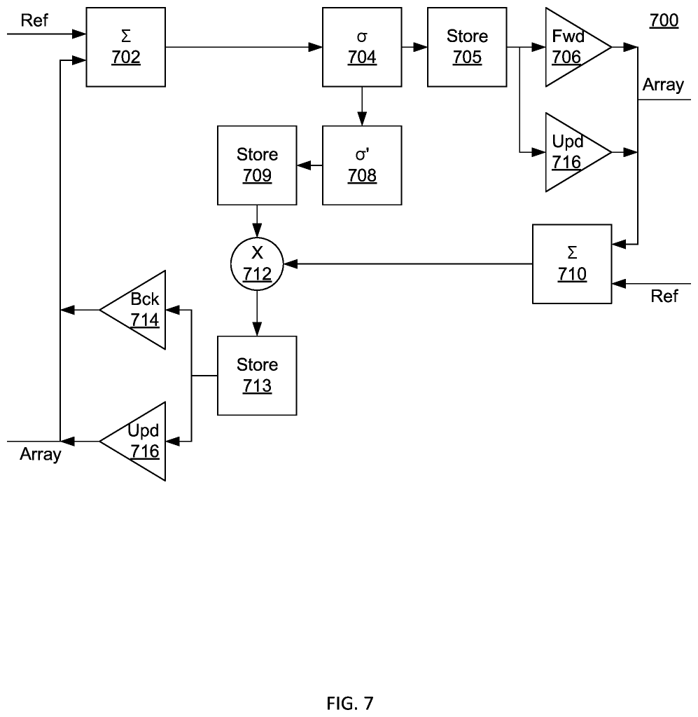

Referring now to FIG. 7, a block diagram of a neuron 700 is shown. This neuron can represent any of the input neurons 602, the hidden neurons 606, or the output neurons 608. It should be noted that FIG. 7 shows components to address all three phases of operation: feed forward, back propagation, and weight update. However, because the different phases do not overlap, there will necessarily be some form of control mechanism within in the neuron 700 to control which components are active. It should therefore be understood that there can be switches and other structures that are not shown in the neuron 700 to handle switching between modes.

In feed forward mode, a difference block 702 determines the value of the input from the array by comparing it to the reference input. This sets both a magnitude and a sign (e.g., + or −) of the input to the neuron 700 from the array. Block 704 performs a computation based on the input, the output of which is stored in storage 705. It is specifically contemplated that block 704 computes a non-linear function and can be implemented as analog or digital circuitry or can be performed in software. The value determined by the function block 704 is converted to a voltage at feed forward generator 706, which applies the voltage to the next array. The signal propagates this way by passing through multiple layers of arrays and neurons until it reaches the final output layer of neurons. The input is also applied to a derivative of the non-linear function in block 708, the output of which is stored in memory 709.

During back propagation mode, an error signal is generated. The error signal can be generated at an output neuron 608 or can be computed by a separate unit that accepts inputs from the output neurons 608 and compares the output to a correct output based on the training data. Otherwise, if the neuron 700 is a hidden neuron 606, it receives back propagating information from the array of weights 604 and compares the received information with the reference signal at difference block 710 to provide a continuously valued, signed error signal. This error signal is multiplied by the derivative of the non-linear function from the previous feed forward step stored in memory 709 using a multiplier 712, with the result being stored in the storage 713. The value determined by the multiplier 712 is converted to a backwards propagating voltage pulse proportional to the computed error at back propagation generator 714, which applies the voltage to the previous array. The error signal propagates in this way by passing through multiple layers of arrays and neurons until it reaches the input layer of neurons 602.

During weight update mode, after both forward and backward passes are completed, each weight 604 is updated proportional to the product of the signal passed through the weight during the forward and backward passes. The update signal generators 716 provide voltage pulses in both directions (though note that, for input and output neurons, only one direction will be available). The shapes and amplitudes of the pulses from update generators 716 are configured to change a state of the weights 604, such that the resistance of the weights 604 is updated.

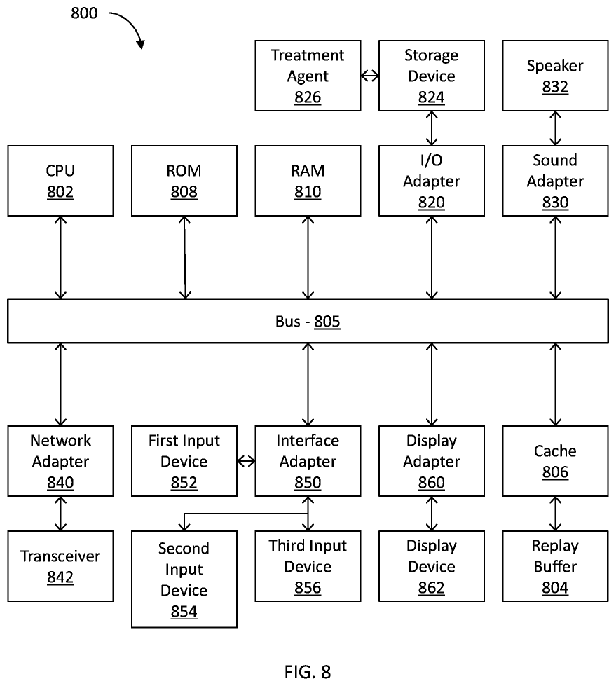

Now referring to FIG. 8, an exemplary processing system 800 to which the present invention may be applied is shown in accordance with one embodiment. The processing system 800 includes at least one processor (CPU) 804 operatively coupled to other components via a system bus 802. A cache 806, a Read Only Memory (ROM) 808, a Random Access Memory (RAM) 810, an input/output (I/O) adapter 820, a sound adapter 830, a network adapter 840, a user interface adapter 850, and a display adapter 860, are operatively coupled to the system bus 805.

A first storage device 824 are operatively coupled to system bus 805 by the I/O adapter 820. The storage device 824 can be any of a disk storage device (e.g., a magnetic or optical disk storage device), a solid state magnetic device, and so forth.

The storage device 824 can include a prediction agent such as, e.g., the treatment agent 826 in communication with the storage device 824. The treatment agent 826 can be loaded from the storage device 824, e.g., into the RAM 810 via the bus 802 for execution by the CPU 802. Thus, the treatment agent 826 can generate actions according to states determine from input to, e.g., the user interface adapter 850 or I/O adapter 820 from, e.g., a patient, a physician, or a measurement device. Objectives corresponding to the patient can be stored in the storage device 824 as well to provide to the value model of the prediction agent. Thus, states provided by the input can be assessed against the objectives even where the goal of, e.g., resolution of a condition, has not yet been achieved.

A replay buffer 804 can be in communication with cache 806 for temporary storage of, e.g., a state and action history for use by the treatment agent 826. The replay buffer 804 can provide a batch of the state and action history via the cache 806 and the bus 805 to CPU 802. The treatment agent 826 can, therefore, call the history for evaluation of actions to generate a suggested action.

A speaker 832 is operatively coupled to system bus 805 by the sound adapter 830. A transceiver 842 is operatively coupled to system bus 802 by network adapter 840. A display device 862 is operatively coupled to system bus 805 by display adapter 860.

A first user input device 852, a second user input device 854, and a third user input device 856 are operatively coupled to system bus 805 by user interface adapter 850. The user input devices 852, 854, and 856 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 852, 854, and 856 can be the same type of user input device or different types of user input devices. The user input devices 852, 854, and 856 are used to input and output information to and from system 800.

Of course, the processing system 800 may also include other elements (not shown), as readily contemplated by one of skill in the art, as well as omit certain elements. For example, various other input devices and/or output devices can be included in processing system 800, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 800 are readily contemplated by one of ordinary skill in the art given the teachings of the present invention provided herein.

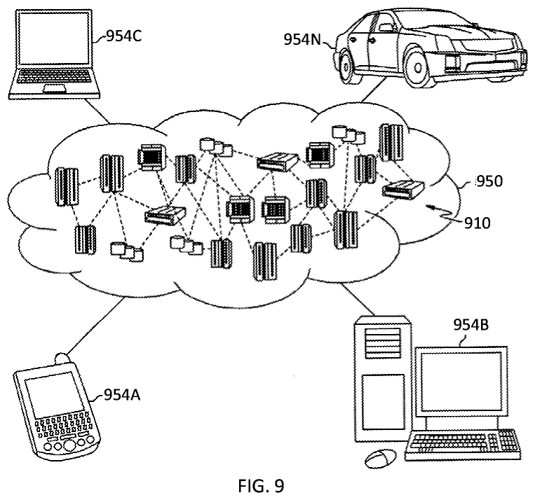

Referring now to FIG. 9, illustrative cloud computing environment 950 is depicted. As shown, cloud computing environment 950 includes one or more cloud computing nodes 910 with which local computing devices used by cloud consumers, such as, for example, personal digital assistant (PDA) or cellular telephone 954A, desktop computer 954B, laptop computer 954C, and/or automobile computer system 954N may communicate. Nodes 910 may communicate with one another. They may be grouped (not shown) physically or virtually, in one or more networks, such as Private, Community, Public, or Hybrid clouds as described hereinabove, or a combination thereof. This allows cloud computing environment 950 to offer infrastructure, platforms and/or software as services for which a cloud consumer does not need to maintain resources on a local computing device. It is understood that the types of computing devices 954A-N shown in FIG. 9 are intended to be illustrative only and that computing nodes 910 and cloud computing environment 950 can communicate with any type of computerized device over any type of network and/or network addressable connection (e.g., using a web browser).

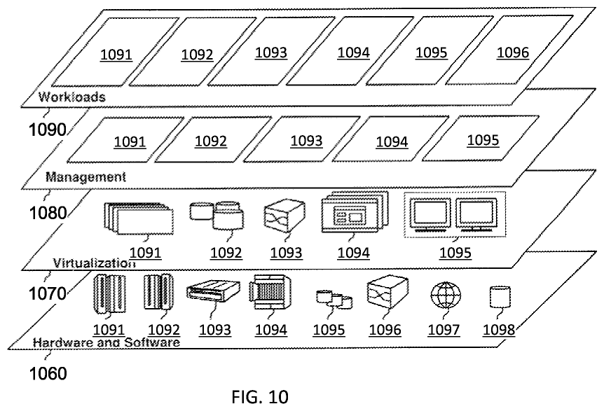

Referring now to FIG. 10, a set of functional abstraction layers provided by cloud computing environment 950 (FIG. 9) is shown. It should be understood in advance that the components, layers, and functions shown in FIG. 10 are intended to be illustrative only and embodiments of the invention are not limited thereto. As depicted, the following layers and corresponding functions are provided:

Hardware and software layer 1060 includes hardware and software components. Examples of hardware components include: mainframes 1061; RISC (Reduced Instruction Set Computer) architecture based servers 1062; servers 1063; blade servers 1064; storage devices 1065; and networks and networking components 1066. In some embodiments, software components include network application server software 1067 and database software 1068.

Virtualization layer 1070 provides an abstraction layer from which the following examples of virtual entities may be provided: virtual servers 1071; virtual storage 1072; virtual networks 1073, including virtual private networks; virtual applications and operating systems 1074; and virtual clients 1075.

In one example, management layer 1080 may provide the functions described below. Resource provisioning 1081 provides dynamic procurement of computing resources and other resources that are utilized to perform tasks within the cloud computing environment. Metering and Pricing 1082 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 1083 provides access to the cloud computing environment for consumers and system administrators. Service level management 1084 provides cloud computing resource allocation and management such that required service levels are met. Service Level Agreement (SLA) planning and fulfillment 1085 provide pre-arrangement for, and procurement of, cloud computing resources for which a future requirement is anticipated in accordance with an SLA.

Workloads layer 1090 provides examples of functionality for which the cloud computing environment may be utilized. Examples of workloads and functions which may be provided from this layer include: mapping and navigation 1091; software development and lifecycle management 1092; virtual classroom education delivery 1093; data analytics processing 1094; transaction processing 1095; and a treatment prediction agent 1096.

The treatment prediction agent 1096 can include, e.g., a state representation model and value model that interacts with patient monitoring systems via processing in the virtualization layer 1070. Thus, data, such as, e.g., patient conditions, can be input into a virtual machine managed in the virtualization layer 1070 according to, e.g., a SLA at the service level management 1084, and stored in the virtual storage 1072. As a result, the treatment prediction agent 1096 can assess changes in states resulting from actions predicted by the treatment prediction agent 1096 to determine whether goals and objectives have been achieved.

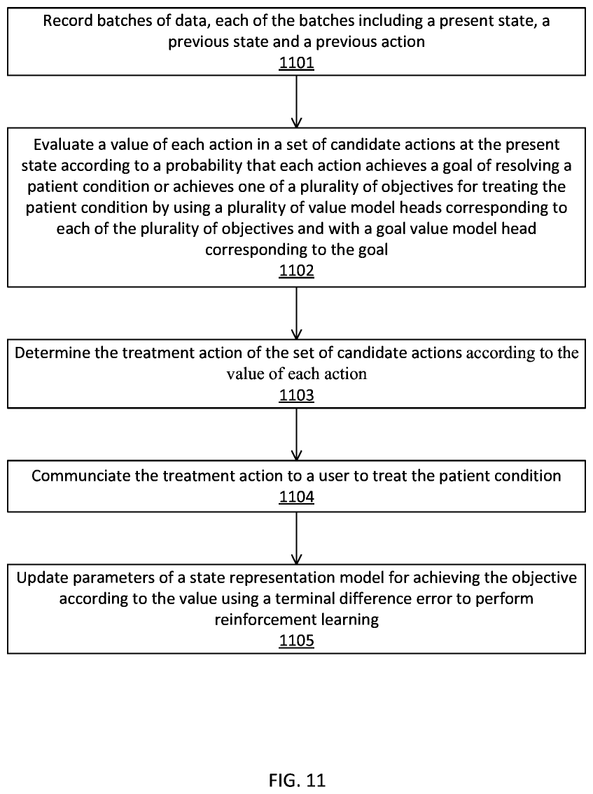

Referring now to FIG. 11, a block/flow diagram showing a system/method generating objective-based treatment actions is illustratively depicted according to an embodiment of the present invention.

At block 1101, record batches of data, each of the batches including a present state, a previous state and a previous action.

At block 1102, evaluate a value of each action in a set of candidate actions at the present state according to a probability that each action achieves a goal of resolving a patient condition or achieves one of a plurality of objectives for treating the patient condition by using a plurality of value model heads corresponding to each of the plurality of objectives and with a goal value model head corresponding to the goal.

At block 1103, determine the treatment action of the set of candidate actions according to the value of each action.

At block 1104, communicate the treatment action to a user to treat the patient condition.

At block 1105, update parameters of a state representation model for achieving the objective according to the value using a terminal difference error to perform reinforcement learning.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for determining a treatment action, the method comprising:
recording batches of data in a replay buffer, each of the batches including a present state, a previous state and a previous action;
evaluating a value of each action in a set of candidate actions at the present state according to a probability that each action achieves a goal of resolving a patient condition or achieves an objective for treating the patient condition by using a value model head corresponding to the goal and the objective;
determining the treatment action of the set of candidate actions according to the value of each action;
communicating the treatment action to a user to treat the patient condition;
determining an error of the value of each action according to whether the previous state achieved by the previous action matches the goal of the objective; and
updating parameters of the value model according to the error.

2. The method as recited in claim 1, further including a value model having a plurality of value model heads, each value model head corresponding to one of a plurality of objectives.

3. The method as recited in claim 2, wherein the value model further includes a goal value model head corresponding to the goal.

4. The method as recited in claim 1, wherein the error includes a temporal difference error to perform reinforcement learning.

5. The method as recited in claim 1, further including determining the present state based on feedback from a condition monitor.

6. The method as recited in claim 1, further including a display to display the treatment action to a user as a recommended treatment.

7. The method as recited in claim 1, wherein the state representation model includes a deep neural network.

8. The method as recited in claim 1, wherein determining the treatment action includes selecting an action of the set of candidate actions that maximizes a product of values corresponding to each of at least one objective and the goal.

9. The method as recited in claim 1, further including evaluating actions until an episode is complete, the episode including a pre-determined time period.

10. A method for generating a treatment action, the method comprising:
recording batches of data, each of the batches including a present state, a previous state and a previous action;
evaluating a value of each action in a set of candidate actions at the present state according to a probability that each action achieves a goal of resolving a patient condition or achieves one of a plurality of objectives for treating the patient condition by using a plurality of value model heads corresponding to each of the plurality of objectives and with a goal value model head corresponding to the goal;
determining the treatment action of the set of candidate actions according to the value of each action;
communicating the treatment action to a user to treat the patient condition; and
updating parameters of a state representation model for achieving the objective according to the value using a terminal difference error to perform reinforcement learning.

11. The method as recited in claim 10, further including determining the present state based on feedback from a condition monitor.

12. The method as recited in claim 10, further including a display to display the treatment action to a user to treat the patient condition.

13. The method as recited in claim 10, wherein the state representation model includes a deep neural network.

14. The method as recited in claim 10, wherein determining the treatment action includes selecting an action of the set of candidate actions that maximizes a product of values corresponding to each of at least one objective and the goal.

15. The method as recited in claim 10, further including evaluating actions until an episode is complete, the episode including a pre-determined time period.

16. A treatment action generation system, comprising:
a replay buffer to record batches of data, each of the batches including a present state, a previous state and a previous action;
a value model head corresponding to an objective for treating a patient condition that retrieves the batches of data to evaluate a value of each action in a set of candidate actions at the present state according to a probability that each action achieves a goal of resolving a patient condition or achieves an objective for treating the patient condition; and
an optimizer to determine the treatment action from the set of candidate actions according to the value of each action and to update parameters of a state representation model for achieving the objective according to the value determined by the value model; and
a connection to communicate the treatment action to a user to treat the patient condition.

17. The system as recited in claim 16, further including a value model with a plurality of value model heads, each of the value model heads corresponding to one of a plurality of objectives.

18. The system as recited in claim 17, wherein the value model further includes a value model head corresponding to the goal.

19. The system as recited in claim 16, wherein the optimizer determines a temporal difference error to update the parameters to perform reinforcement learning.

20. The system as recited in claim 16, further including a condition monitor to provide patient condition feedback to determine the present state.
